(19) United States
(12) Patent Application Publication
(10) Pub. No.: US 2025/0262391 A1
(43) Pub. Date: Aug. 21, 2025

(54) Nebulizer

(71) Applicant: SHIMADZU CORPORATION, Kyoto-shi (JP)

(72) Inventors: Hiroki MIYASHIRO, Kyoto-shi (JP); Manabu UEDA, Kyoto-shi (JP); Kazuki KOMATSU, Kyoto-shi (JP); Shinya MATSUURA, Kyoto-shi (JP)

(21) Appl. No.: 19/006,776

(22) Filed: Dec. 31, 2024

(30) Foreign Application Priority Data
Feb. 16, 2024 (JP) 2024-021685

(51) Int. Cl. A61M 11/02

(52) U.S. Cl. CPC A61M 11/02

(57) ABSTRACT

A nebulizer includes: a nozzle portion provided with an ejection port through which gas is ejected; and a capillary having a tip end portion disposed to protrude from the ejection port. A plurality of protruding portions protruding inward in a radial direction of the ejection port are arranged side by side in a circumferential direction on a portion of an inner circumferential surface of the nozzle portion, the portion of the inner circumferential surface defining the ejection port. The plurality of protruding portions are provided to define an inscribed circle. The capillary passes through inside the inscribed circle.

### CROSS REFERENCE TO RELATED APPLICATIONS

This nonprovisional application is based on Japanese Patent Application No. 2024-021685 filed on Feb. 16, 2024 with the Japan Patent Office, the entire contents of which are hereby incorporated by reference.

### BACKGROUND OF THE INVENTION

### Field of the Invention

The present disclosure relates to a nebulizer.

### Description of the Background Art

As a conventional nebulizer for an ion source, US Patent Publication No. 2021/0398789 discloses a configuration in which a nebulizer outlet portion forming an outlet of the nebulizer is attached to a tip end of a hollow cylindrical body through which gas flows.

By way of example, the nebulizer outlet portion is constituted of a plurality of parts. Specifically, the nebulizer outlet portion includes: an inlet-side member constituting an inlet end; and an outlet-side member constituting an outlet end. The inlet-side member is attached to a tip end of the hollow cylindrical body, and the outlet-side member is attached downstream of the inlet-side member.

The inlet-side member is provided with a first channel and a second channel extending through the inlet-side member in its axial direction. The first channel is provided along a central axis of the nebulizer outlet portion, and a capillary passes through the first channel. The second channel communicates with the space inside the hollow cylindrical body and serves as a flow path through which the gas introduced into the hollow cylindrical body flows.

The outlet-side member has a shape tapered toward the outlet. A space communicating with the second channel extends between the inner surface of the outlet-side member and the tip end of the inlet-side member. The outlet-side member has a tip end provided with an outlet opening though which the tip end side of the capillary passes. Also, a gap is provided between the inner circumferential surface of the outlet-side member that defines the outlet opening and the tip end of the capillary. The gas is ejected through the gap.

As another example, the nebulizer outlet portion is formed of a single member. In this case, in a cross section parallel to the axial direction, on the upstream side of the outlet opening, a substantially V-shaped space is provided, a first channel through which the capillary passes is provided coaxially with the central axis of the outlet opening, and a second channel communicating with the V-shaped space is provided.

### SUMMARY OF THE INVENTION

In the disclosure in US Patent Publication No. 2021/0398789, in the case where the nebulizer outlet portion is constituted of a plurality of parts as described above, the plurality of parts need to be coaxially attached, which requires accuracy in each of the parts and thereby makes it difficult to perform machining. Further, in the case where the nebulizer outlet portion is formed of a single member as described above, the additive manufacturing process needs to be adopted in order to provide the space portion, the first channel, and the second channel, each of which has a complicated shape. This makes it difficult to fabricate a product with general-purpose tools and methods such as an end mill and electric discharge machining.

Further, in the configuration disclosed in the above-mentioned US Patent Publication No. 2021/0398789, the approximate position of the capillary is determined by the first channel located away from the capillary on the upstream side of the nebulizer outlet portion. This causes a concern that the capillary may be disposed eccentrically from the center of the outlet opening. In this case, the gas blown out from the outlet opening causes unevenness in the spray shape of the droplets sprayed from the capillary.

The present disclosure has been made in view of the above-described problems, and an object thereof is to provide a nebulizer that allows a substantially uniform spray to be emitted from an ejection port in a simple configuration.

A nebulizer according to a first aspect of the present disclosure includes: a nozzle portion provided with an ejection port through which gas is ejected; and a capillary having a tip end portion disposed to protrude from the ejection port. A plurality of protruding portions protruding inward in a radial direction of the ejection port are arranged side by side in a circumferential direction on a portion of an inner circumferential surface of the nozzle portion, the portion of the inner circumferential surface defining the ejection port. The plurality of protruding portions are provided to define an inscribed circle. The capillary passes through inside the inscribed circle.

According to the above-described configuration, by a simple configuration of the plurality of protruding portions provided on the inner circumferential surface of the nozzle portion, the center of the capillary can be suppressed from being disposed at a position eccentric from the center of the inscribed circle. Further, since the plurality of protruding portions serving to determine the position of the capillary are provided at the ejection port, the displacement of the tip end position of the capillary can be effectively suppressed. This consequently makes it possible to suppress unevenness in the spray shape of the droplets sprayed from the capillary, so that the droplets can be substantially uniformly sprayed from the ejection port.

A nebulizer according to a second aspect of the present disclosure includes: a nozzle portion provided with an ejection port through which gas is ejected; and a capillary having a tip end portion disposed to protrude from the ejection port. A portion of an inner circumferential surface of the nozzle portion is provided in a circular shape when viewed in an axial direction of the ejection port, the portion of the inner circumferential surface defining the ejection port. The capillary has an outer shape having a plurality of corner portions. The plurality of corner portions are located inside the circular shape when viewed in the axial direction.

According to the above-described configuration, by a simple configuration in which the ejection port is formed in a circular cylindrical shape and the capillary has an outer shape having a plurality of corner portions, the center of the capillary can be suppressed from being disposed at a position eccentric from the center of the circular ejection port. Further, since the positions of the plurality of corner portions are determined by the ejection port, the displacement of the tip end position of the capillary can be effectively suppressed. This consequently makes it possible to suppress unevenness in the spray shape of the droplets sprayed from the capillary, so that the droplets can be substantially uniformly sprayed from the ejection port.

The foregoing and other objects, features, aspects, and advantages of the present invention will become apparent from the following detailed description of the present invention when taken in conjunction with the accompanying drawings.

### BRIEF DESCRIPTION OF THE DRAWINGS

FIG. 1 is a schematic cross-sectional view of a nebulizer according to a first embodiment.

FIG. 2 is an enlarged perspective view showing an ejection port of the nebulizer according to the first embodiment and the vicinity of the ejection port.

FIG. 3 is a diagram showing an end surface on a tip end side of a nozzle portion according to the first embodiment as viewed in an axial direction.

FIG. 4 is a diagram showing an end surface on a tip end side of a nozzle portion according to a first modification as viewed in the axial direction.

FIG. 5 is a diagram showing an end surface on a tip end side of a nozzle portion according to a second modification as viewed in the axial direction.

FIG. 6 is a diagram showing an end surface on a tip end side of a nozzle portion according to a third modification as viewed in the axial direction.

FIG. 7 is a diagram showing an end surface on a tip end side of a nozzle portion according to a fourth modification as viewed in the axial direction.

FIG. 8 is an enlarged perspective view showing an ejection port of a nozzle portion according to a second embodiment and the vicinity of the ejection port.

FIG. 9 is a diagram showing a tip end portion of a capillary according to the second embodiment as viewed in the axial direction.

FIG. 10 is a diagram showing a tip end portion of a capillary according to a fifth modification as viewed in the axial direction.

FIG. 11 is a diagram showing a tip end portion of a capillary according to a sixth modification as viewed in the axial direction.

FIG. 12 is a diagram showing a tip end portion of a capillary according to a seventh modification as viewed in the axial direction.

FIG. 13 is a diagram showing a tip end portion of a capillary according to an eighth modification as viewed in the axial direction.

### DESCRIPTION OF THE PREFERRED EMBODIMENTS

Hereinafter, embodiments of the present disclosure will be described in detail with reference to the accompanying drawings. In the embodiments described below, the same or corresponding portions are denoted by the same reference characters in the drawings, and the description thereof will not be repeated.

### First Embodiment

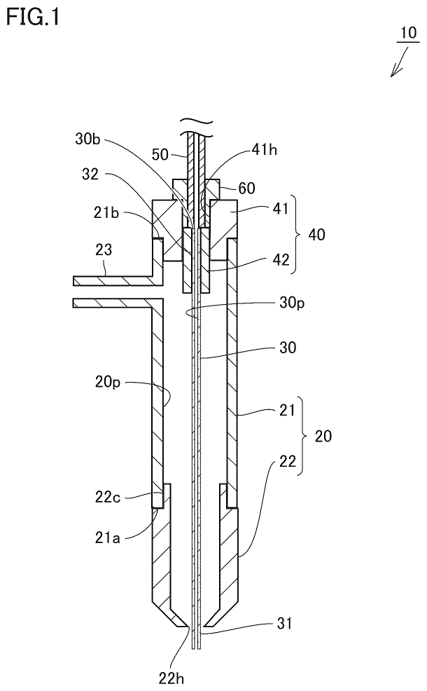

FIG. 1 is a schematic cross-sectional view of a nebulizer according to the first embodiment. The following describes a nebulizer 10 according to the first embodiment with reference to FIG. 1.

Nebulizer 10 is used, for example, in an ion analyzer such as a mass spectrometer and an ion mobility spectrometer for performing ionization.

Nebulizer 10 includes a body portion 20, a capillary 30, and a capillary holding portion 40. Body portion 20 has a substantially hollow cylindrical shape and constitutes a double tube structure together with capillary 30 disposed inside body portion 20. In other words, body portion 20 is disposed to surround capillary 30 in the circumferential direction. A gas flow path 20p through which gas flows is provided inside body portion 20. A flow path 30p through which a liquid such as a sample solution flows is provided in capillary 30.

Body portion 20 includes a hollow cylindrical member 21 and a nozzle portion 22. Hollow cylindrical member 21 has a circular hollow cylindrical shape. Hollow cylindrical member 21 extends in an axial direction. Hollow cylindrical member 21 has one end 21a and the other end 21b. One end 21a is located downstream from the other end 21b in the direction in which the above-mentioned gas flows. A gas introduction portion 23 is provided on the other end 21b side of hollow cylindrical member 21. Gas introduction portion 23 extends in the direction intersecting with the axial direction of hollow cylindrical member 21.

Nozzle portion 22 is formed such that its tip end side has a substantially conical cylindrical shape and its base end side has a circular hollow cylindrical shape. The tip end of nozzle portion 22 is provided with an ejection port 22h through which gas is ejected. The gas introduced from gas introduction portion 23 flows through gas flow path 20p and thereafter is ejected from ejection port 22h.

Nozzle portion 22 is fixed to the one end 21a side of hollow cylindrical member 21. The base end portion of nozzle portion 22 is provided with an engagement portion 22c. Engagement portion 22c engages with the one end 21a side of hollow cylindrical member 21, so that nozzle portion 22 is fixed.

Capillary 30 has a hollow cylindrical shape that is smaller in outer diameter and inner diameter than hollow cylindrical member 21. Capillary 30 has a tip end portion 31 and a base end portion 32. Capillary 30 is disposed such that tip end portion 31 protrudes from ejection port 22h. Capillary 30 is mostly located inside body portion 20. Base end portion 32 of capillary 30 is held by capillary holding portion 40.

Capillary holding portion 40 includes a first member 41 and a second member 42. First member 41 is fixed to the other end 21b of hollow cylindrical member 21. More specifically, first member 41 is fixed in the state in which its tip end side is partially inserted into the other end 21b side of hollow cylindrical member 21. First member 41 has a through hole 41h. The central axis of through hole 41h substantially coincides with the central axis of hollow cylindrical member 21.

A joint 60 having a pipe 50 inserted thereinto is fixed to the base end side of through hole 41h. Second member 42 is fixed to the tip end side of through hole 41h. Second member 42 has a hollow cylindrical shape. Base end portion 32 of capillary 30 is inserted into the inside of second member 42.

As described above, base end portion 32 of capillary 30 is inserted into the inside of second member 42 inserted into the through hole of first member 41 fixed to the other end 21b side of hollow cylindrical member 21. Thereby, the base end portion 32 side of capillary 30 is held by capillary holding portion 40.

Note that capillary 30 is inserted into second member 42 such that flow path 30p communicates with the inner space of pipe 50. A sample solution is supplied to pipe 50, and thereby, a sample is supplied to capillary 30.

The sample supplied to capillary 30 passes through flow path 30p and is then sprayed in a mist from the tip end of capillary 30 by the gas ejected from ejection port 22h. The gas promotes vaporization of the droplets sprayed from the tip end of capillary 30 to promote ionization.

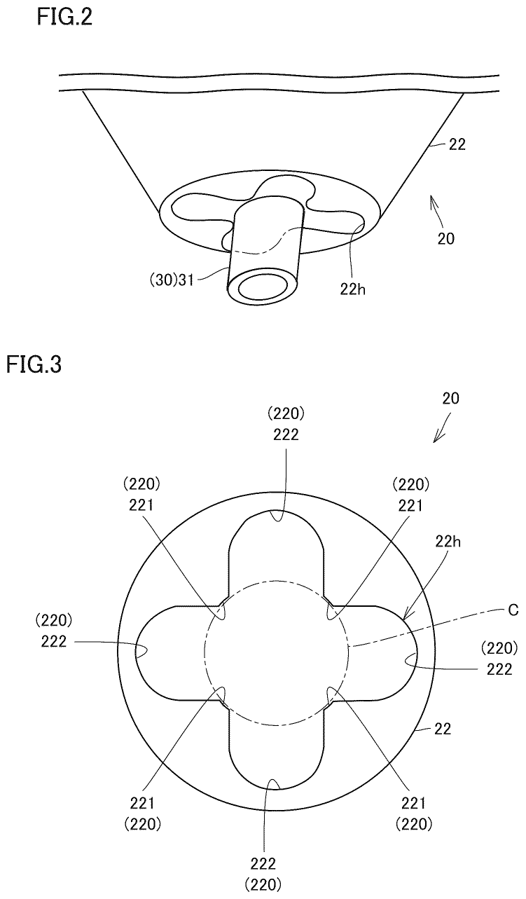

FIG. 2 is an enlarged perspective view showing an ejection port of the nebulizer according to the first embodiment and the vicinity of the ejection port. FIG. 3 is a diagram showing an end surface on the tip end side of the nozzle portion according to the first embodiment as viewed in the axial direction.

As shown in FIGS. 2 and 3, a plurality of protruding portions 221 protruding inward in the radial direction of ejection port 22h are arranged side by side in the circumferential direction on an inner circumferential surface 220 of nozzle portion 22, inner circumferential surface 220 defining ejection port 22h. The plurality of protruding portions 221 are provided so as to define an inscribed circle C. The plurality of protruding portions 221 are provided such that their top surfaces draw an arc shape when viewed in the axial direction of (in the direction of the normal to) ejection port 22h. Each of the top surfaces faces the central axis of ejection port 22h. The plurality of protruding portions 221 are arranged side by side at prescribed intervals in the circumferential direction. More specifically, the plurality of protruding portions 221 include four protruding portions that are arranged at a 90-degree pitch.

Further, inner circumferential surface 220 of nozzle portion 22 that defines ejection port 22h has a plurality of bulging portions 222. The plurality of bulging portions 222 bulge outward of inscribed circle C. The plurality of bulging portions 222 are arranged side by side at prescribed intervals in the circumferential direction. The plurality of bulging portions 222 include four bulging portions that are arranged at a 90-degree pitch. By arranging bulging portions 222 at a prescribed pitch, the gas can be ejected substantially uniformly from ejection port 22h around capillary 30. Each bulging portion 222 has a shape obtained by dividing an elongated hole into two parts along the minor axis direction when viewed in the axial direction. Protruding portion 221 is disposed between bulging portions 222 adjacent to each other in the circumferential direction. Bulging portion 222 has an opening opened at inscribed circle C, and the width of this opening is smaller than the outer diameter of capillary 30. In other words, the interval between protruding portions 221 adjacent to each other in the circumferential direction is smaller than the outer diameter of capillary 30. This makes it possible to prevent capillary 30 from entering bulging portion 222.

Capillary 30 passes through inscribed circle C defined by the plurality of protruding portions 221 as described above. The diameter of inscribed circle C is substantially equal to the outer diameter of capillary 30.

Capillary 30 is positioned inside inscribed circle C such that the center of inscribed circle C substantially coincides with the center of capillary 30 by the plurality of protruding portions 221. Thereby, by the simple configuration of the plurality of protruding portions 221 provided on inner circumferential surface 220 of nozzle portion 22, the center of capillary 30 can be suppressed from being disposed at a position eccentric from the center of inscribed circle C.

Further, since the plurality of protruding portions 221 serving to determine the position of capillary 30 are provided at ejection port 22h, the displacement of the tip end position of capillary 30 can be effectively suppressed. More specifically, the plurality of protruding portions 221 serving to determine the position of capillary 30 are provided at ejection port 22h that is the outlet end of gas flow path 20p. Accordingly, the central axis of capillary 30 can be positioned to be substantially coaxial with the central axis of ejection port 22h at the outlet end with high accuracy as compared with the configuration in which capillary 30 is aligned at some intermediate position of gas flow path 20p. This consequently makes it possible to suppress unevenness in the spray shape of the droplets sprayed from capillary 30, so that the droplets can be substantially uniformly sprayed from ejection port 22h.

Further, ejection port 22h provided at the end surface of nozzle portion 22 located at the tip end has a fixed shape along the axial direction of nozzle portion 22. Thus, the configuration of nozzle portion 22 can be simplified, and ejection port 22h can be fabricated by general-purpose tools and methods such as an end mill and electric discharge machining. This eliminates the need to employ a special manufacturing method such as additive manufacturing, so that nozzle portion 22 and therefore a nebulizer can be manufactured easily and inexpensively.

In addition, a member (a positioning member) for positioning tip end portion 31 of capillary 30 is formed of a single nozzle portion 22. Thereby, as compared with the case where the positioning member is formed of a plurality of divided parts, the processing accuracy can be improved, so that the accuracy in positioning capillary 30 can be enhanced.

### First Modification

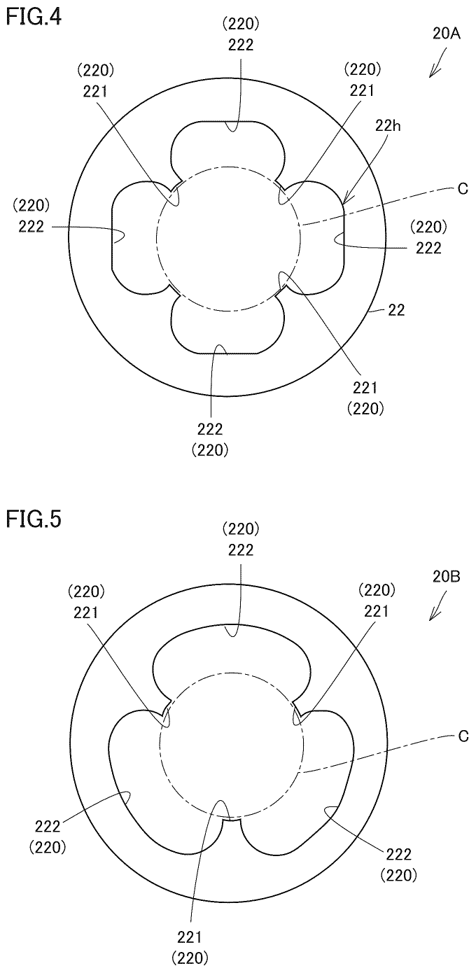

FIG. 4 is a diagram showing an end surface on a tip end side of a nozzle portion according to the first modification as viewed in the axial direction. The following describes a nebulizer according to the first modification with reference to FIG. 4.

The nebulizer according to the first modification is different from the nebulizer according to the first embodiment in the shape of a tip end of a nozzle portion 22A and the shape of ejection port 22h. Other configurations are substantially the same.

Also in the first modification, a plurality of protruding portions 221 arranged side by side in the circumferential direction on inner circumferential surface 220 of nozzle portion 22 that defines ejection port 22h are provided so as to define inscribed circle C, and capillary 30 passes through inscribed circle C. Four protruding portions 221 are provided and arranged at a 90-degree pitch.

Further, four bulging portions 222 are arranged at a 90-degree pitch in the circumferential direction. Each of four bulging portions 222 has a track shape whose minor axis is substantially in parallel to the radial direction when viewed in the axial direction. Protruding portion 221 is disposed between bulging portions 222 adjacent to each other in the circumferential direction.

Even in the case of the configuration as described above, the nebulizer according to the first modification can achieve substantially the same effect as that achieved by the nebulizer according to the first embodiment.

### Second Modification

FIG. 5 is a diagram showing an end surface on a tip end side of a nozzle portion according to the second modification as viewed in the axial direction. The following describes a nebulizer according to the second modification with reference to FIG. 5.

The nebulizer according to the second modification is different from the nebulizer according to the first embodiment in the shape of a tip end of a nozzle portion 22B and the shape of ejection port 22h. Other configurations are substantially the same.

Also in the second modification, a plurality of protruding portions 221 arranged side by side in the circumferential direction on inner circumferential surface 220 of nozzle portion 22 that defines ejection port 22h are provided so as to define inscribed circle C, and capillary 30 passes through inscribed circle C. Three protruding portions 221 are provided and arranged at a 120-degree pitch.

Three bulging portions 222 are arranged at a 120-degree pitch in the circumferential direction. Each of three bulging portions 222 has an elongated hole shape extending in a substantially arc shape when viewed in the axial direction. Protruding portion 221 is disposed between bulging portions 222 adjacent to each other in the circumferential direction.

Even in the case of the configuration as described above, the nebulizer according to the second modification can achieve substantially the same effect as that achieved by the nebulizer according to the first embodiment.

### Third Modification

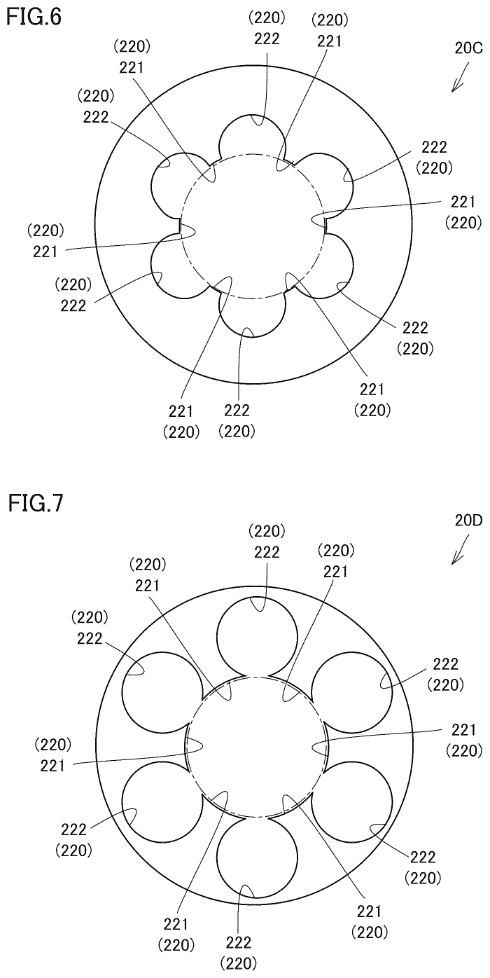

FIG. 6 is a diagram showing an end surface on a tip end side of a nozzle portion according to the third modification as viewed in the axial direction. The following describes a nebulizer according to the third modification with reference to FIG. 6.

The nebulizer according to the third modification is different from the nebulizer according to the first embodiment in the shape of a tip end of a nozzle portion 22C and the shape of ejection port 22h. Other configurations are substantially the same.

Also in the third modification, a plurality of protruding portions 221 arranged side by side in the circumferential direction on inner circumferential surface 220 of nozzle portion 22 that defines ejection port 22h are provided so as to define inscribed circle C, and capillary 30 passes through inscribed circle C. Six protruding portions 221 are provided and arranged at a 60-degree pitch.

Further, six bulging portions 222 are arranged at a 60-degree pitch in the circumferential direction, and each of six bulging portions 222 has a substantially semicircular shape when viewed in the axial direction. Protruding portion 221 is disposed between bulging portions 222 adjacent to each other in the circumferential direction.

Even in the case of the configuration as described above, the nebulizer according to the third modification can achieve substantially the same effect as that achieved by the nebulizer according to the first embodiment.

### Fourth Modification

FIG. 7 is a diagram showing an end surface on a tip end side of a nozzle portion according to the fourth modification as viewed in the axial direction. The following describes a nebulizer according to the fourth modification with reference to FIG. 7.

The nebulizer according to the fourth modification is different from the nebulizer according to the first embodiment in the shape of a tip end of a nozzle portion 22D and the shape of ejection port 22h. Other configurations are substantially the same.

Also in the fourth modification, a plurality of protruding portions 221 arranged side by side in the circumferential direction on inner circumferential surface 220 of nozzle portion 22 that defines ejection port 22h are provided so as to define inscribed circle C, and capillary 30 passes through inscribed circle C. Six protruding portions 221 are provided and arranged at a 60-degree pitch.

Further, six bulging portions 222 are arranged at a 60-degree pitch in the circumferential direction, and each of six bulging portions 222 has a substantially circular shape when viewed in the axial direction. Protruding portion 221 is disposed between bulging portions 222 adjacent to each other in the circumferential direction.

Even in the case of the configuration as described above, the nebulizer according to the fourth modification can achieve substantially the same effect as that achieved by the nebulizer according to the first embodiment.

### Second Embodiment

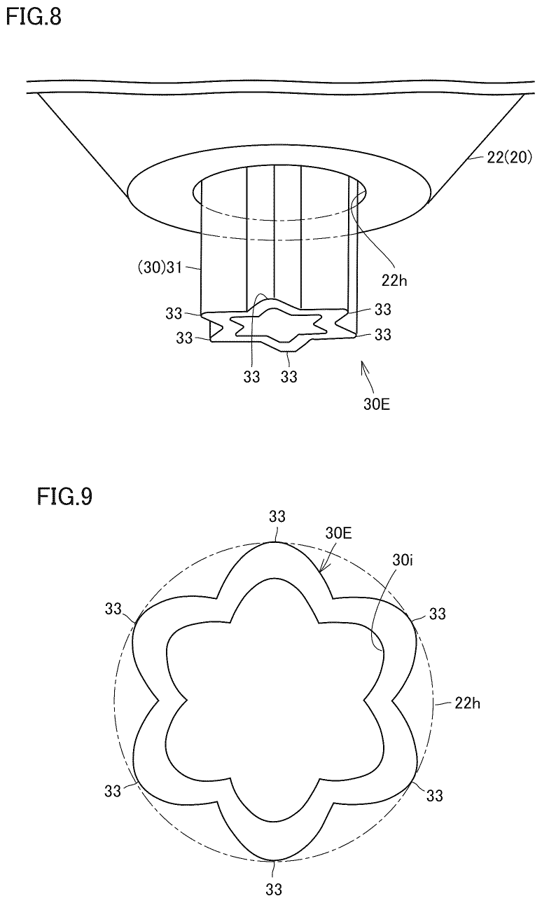

FIG. 8 is an enlarged perspective view showing an ejection port of a nozzle portion according to the second embodiment and the vicinity of the ejection port. FIG. 9 is a diagram showing a tip end portion of a capillary according to the second embodiment as viewed in the axial direction. The following describes a nebulizer according to the second embodiment with reference to FIGS. 8 and 9.

As shown in FIGS. 8 and 9, the nebulizer according to the second embodiment is different from the nebulizer according to the first embodiment in the shape of a capillary 30E, the shape of a tip end of a nozzle portion 22E, and the shape of ejection port 22h. Other configurations are substantially the same.

In the second embodiment, ejection port 22h is provided in a circular shape when viewed in the axial direction of ejection port 22h. In other words, when viewed in the axial direction, a portion of the inner circumferential surface of nozzle portion 22 that defines ejection port 22h is provided in a circular shape.

Further, in capillary 30E having an outer shape having a plurality of corner portions 33, when viewed in the axial direction, the outer shape of capillary 30E is similar to the shape of an inner circumferential surface 30i of the capillary that defines flow path 30p.

Specifically, capillary 30E has a polygonal hollow cylindrical shape. More specifically, capillary 30E has a dodecagonal hollow cylindrical shape whose sides are curved so as to bulge outward. In the illustrated example, the plurality of corner portions 33 are rounded but may not be rounded. The plurality of corner portions 33 are arranged at a prescribed pitch in the circumferential direction. Thereby, the gas can be ejected substantially uniformly from ejection port 22h around capillary 30. When viewed in the axial direction, the plurality of corner portions 33 are disposed inside the above-mentioned circular shape formed by the inner circumferential surface of nozzle portion 22.

Capillary 30E is positioned inside the above-mentioned circular shape by the plurality of corner portions 33 such that the center of the circular shape substantially coincides with the center of capillary 30E. Thereby, in a simple configuration in which ejection port 22h is formed in a circular cylindrical shape and capillary 30 has an outer shape having a plurality of corner portions, the center of capillary 30E can be suppressed from being disposed at a position eccentric from the center of circular ejection port 22h.

Further, since the positions of the plurality of corner portions 33 are determined by ejection port 22h, the displacement of the tip end position of the capillary can be effectively suppressed. This consequently makes it possible to suppress unevenness in the spray shape of the droplets sprayed from the capillary, so that the droplets can be substantially uniformly sprayed from the ejection port.

As described above, the nebulizer according to the second embodiment can achieve substantially the same effect as that achieved by the nebulizer according to the first embodiment.

### Fifth Modification

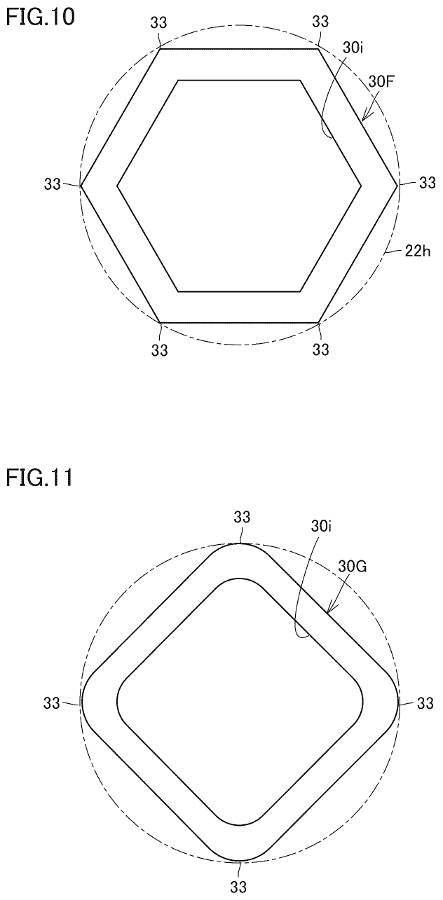

FIG. 10 is a diagram showing a tip end portion of a capillary according to the fifth modification as viewed in the axial direction. The following describes a nebulizer according to the fifth modification with reference to FIG. 10.

The nebulizer according to the fifth modification is different from the nebulizer according to the second embodiment in the shape of a capillary 30F. Other configurations are substantially the same.

Also in the fifth modification, capillary 30F has an outer shape having a plurality of corner portions 33 and specifically has a hexagonal hollow cylindrical shape. The plurality of corner portions 33 are disposed inside the above-mentioned circular shape formed by the inner circumferential surface of nozzle portion 22.

Even in the case of the configuration as described above, the nebulizer according to the fifth modification can achieve substantially the same effect as that achieved by the nebulizer according to the second embodiment.

### Sixth Modification

FIG. 11 is a diagram showing a tip end portion of a capillary according to the sixth modification as viewed in the axial direction. The following describes a nebulizer according to the sixth modification with reference to FIG. 11.

The nebulizer according to the sixth modification is different from the nebulizer according to the second embodiment in the shape of a capillary 30G. Other configurations are substantially the same.

Also in the sixth modification, capillary 30G has an outer shape having a plurality of corner portions 33 and specifically has a quadrangular hollow cylindrical shape. The plurality of corner portions 33 are disposed inside the above-mentioned circular shape formed by the inner circumferential surface of nozzle portion 22.

Even in the case of the configuration as described above, the nebulizer according to the sixth modification can achieve substantially the same effect as that achieved by the nebulizer according to the second embodiment.

### Seventh Modification

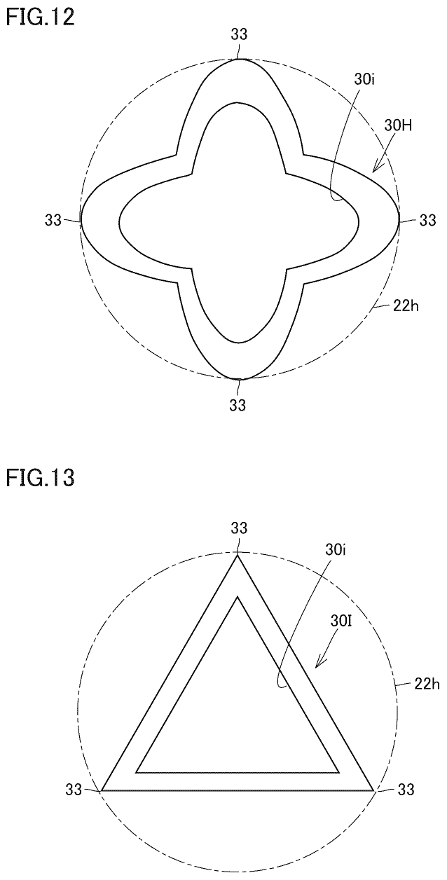

FIG. 12 is a diagram showing a tip end portion of a capillary according to the seventh modification as viewed in the axial direction. The following describes a nebulizer according to the seventh modification with reference to FIG. 12.

The nebulizer according to the seventh modification is different from the nebulizer according to the second embodiment in the shape of a capillary 30H. Other configurations are substantially the same.

Also in the seventh modification, capillary 30H has an outer shape having a plurality of corner portions 33 and specifically has an octagonal hollow cylindrical shape whose sides are curved so as to bulge outward. The plurality of corner portions 33 are disposed inside the above-mentioned circular shape formed by the inner circumferential surface of nozzle portion 22.

Even in the case of the configuration as described above, the nebulizer according to the seventh modification can achieve substantially the same effect as that achieved by the nebulizer according to the second embodiment.

### Eighth Modification

FIG. 13 is a diagram showing a tip end portion of a capillary according to the eighth modification as viewed in the axial direction. The following describes a nebulizer according to the eighth modification with reference to FIG. 13.

The nebulizer according to the eighth modification is different from the nebulizer according to the second embodiment in the shape of a capillary 30I. Other configurations are substantially the same.

Also in the eighth modification, capillary 30I has an outer shape having a plurality of corner portions 33 and specifically has a triangular hollow cylindrical shape. The plurality of corner portions 33 are disposed inside the above-mentioned circular shape formed by the inner circumferential surface of nozzle portion 22.

Even in the case of the configuration as described above, the nebulizer according to the eighth modification can achieve substantially the same effect as that achieved by the nebulizer according to the second embodiment.

### Additional Aspects

### Configuration 1

A nebulizer includes:
a nozzle portion provided with an ejection port through which gas is ejected; and
a capillary having a tip end portion disposed to protrude from the ejection port, wherein
a plurality of protruding portions protruding inward in a radial direction of the ejection port are arranged side by side in a circumferential direction on a portion of an
inner circumferential surface of the nozzle portion, the portion of the inner circumferential surface defining the ejection port,
the plurality of protruding portions are provided to define an inscribed circle, and
the capillary passes through inside the inscribed circle.

### Configuration 2

The nebulizer according to Configuration 1, wherein the plurality of protruding portions are three or more in number.

### Configuration 3

The nebulizer according to Configuration 1 or 2, wherein the plurality of protruding portions are arranged side by side at prescribed intervals in the circumferential direction.

### Configuration 4

The nebulizer according to any one of Configurations 1 to 3, wherein an interval between protruding portions adjacent to each other in the circumferential direction among the plurality of protruding portions is smaller than an outer diameter of the capillary.

### Configuration 5

The nebulizer according to any one of Configurations 1 to 4, wherein
the portion of the inner circumferential surface of the nozzle portion that defines the ejection port has a plurality of bulging portions that bulge outward of the inscribed circle,
the plurality of bulging portions are arranged side by side in the circumferential direction, and
each of the protruding portions is located between bulging portions adjacent to each other in the circumferential direction among the plurality of bulging portions.

### Configuration 6

A nebulizer includes:
a nozzle portion provided with an ejection port through which gas is ejected; and
a capillary having a tip end portion disposed to protrude from the ejection port; wherein
a portion of an inner circumferential surface of the nozzle portion is provided in a circular shape when viewed in an axial direction of the ejection port, the portion of
the inner circumferential surface defining the ejection port,
the capillary has an outer shape having a plurality of corner portions, and the plurality of corner portions are located inside the circular shape when viewed in the axial direction.

### Configuration 7

The nebulizer according to Configuration 6, wherein
the capillary includes a flow path through which a liquid sample flows, and
when viewed in the axial direction, an outer shape of the capillary is similar to a shape of an inner circumferential surface of the capillary, the inner circumferential surface defining the flow path.

Although the embodiments of the present invention have been described, it should be understood that the embodiments disclosed herein are illustrative and not restrictive in every respect. The scope of the present invention is defined by the terms of the claims, and is intended to include any modifications within the meaning and scope equivalent to the terms of the claims.

What is claimed is:

1. A nebulizer comprising:
a nozzle portion provided with an ejection port through which gas is ejected; and
a capillary having a tip end portion disposed to protrude from the ejection port, wherein
a plurality of protruding portions protruding inward in a radial direction of the ejection port are arranged side by side in a circumferential direction on a portion of an inner circumferential surface of the nozzle portion, the portion of the inner circumferential surface defining the ejection port,
the plurality of protruding portions are provided to define an inscribed circle, and
the capillary passes through inside the inscribed circle.

2. The nebulizer according to claim 1, wherein the plurality of protruding portions are three or more in number.

3. The nebulizer according to claim 1, wherein the plurality of protruding portions are arranged side by side at prescribed intervals in the circumferential direction.

4. The nebulizer according to claim 1, wherein an interval between protruding portions adjacent to each other in the circumferential direction among the plurality of protruding portions is smaller than an outer diameter of the capillary.

5. The nebulizer according to claim 1, wherein
the portion of the inner circumferential surface of the nozzle portion that defines the ejection port has a plurality of bulging portions that bulge outward of the inscribed circle,
the plurality of bulging portions are arranged side by side in the circumferential direction, and
each of the protruding portions is located between bulging portions adjacent to each other in the circumferential direction among the plurality of bulging portions.

6. A nebulizer comprising:
a nozzle portion provided with an ejection port through which gas is ejected; and
a capillary having a tip end portion disposed to protrude from the ejection port; wherein
a portion of an inner circumferential surface of the nozzle portion is provided in a circular shape when viewed in an axial direction of the ejection port, the portion of
the inner circumferential surface defining the ejection port,
the capillary has an outer shape having a plurality of corner portions, and
the plurality of corner portions are located inside the circular shape when viewed in the axial direction.

7. The nebulizer according to claim 6, wherein
the capillary includes a flow path through which a liquid sample flows, and
when viewed in the axial direction, an outer shape of the capillary is similar to a shape of an inner circumferential surface of the capillary, the inner circumferential surface defining the flow path.
